(19) United States
(12) Patent Application Publication
(10) Pub. No.: US 2014/0378881 A1
(43) Pub. Date: Dec. 25, 2014

(54) Lower Leg Orthosis

(71) Applicant: Helmut Wagner, Duderstadt (DE)

(72) Inventor: Helmut Wagner, Duderstadt (DE)

(21) Appl. No.: 14/480,676

(22) Filed: Sep. 9, 2014

Related U.S. Application Data
(63) Continuation of application No. PCT/EP2013/054338, filed on Mar. 5, 2013.

(30) Foreign Application Priority Data
Mar. 9, 2012 (DE) 10 2012 102 012.4

(51) Int. Cl. A61F 5/01

(52) U.S. Cl. CPC A61F 5/0127
USPC 602/16

(57) ABSTRACT

The invention relates to a lower leg orthosis. The lower leg orthosis is formed with a foot part, a pivot joint, a coupling element, a supporting element and a retaining device in the form of a band. According to the invention it is proposed that between the coupling element and the supporting element a degree of freedom of movement is present, by means of which a rubbing of the lower leg orthosis on a lower leg of the person wearing the lower leg orthosis is at least partially alleviated.

### CROSS REFERENCE TO RELATED APPLICATIONS

This application is a Continuation of International Application PCT/EP2013/054338 with an International Filing Date of Mar. 5, 2013 and claiming priority to German Patent Application No. 10 2012 102 012.4 entitled “Unterschenkelorthese”, filed on Mar. 9, 2012.

### TECHNICAL FIELD OF THE INVENTION

The invention relates to a lower leg orthosis. In particular for correcting a leg deformity, such as a bow-leg or a knock-knee, lower leg orthoses comprise a foot part and a coupling element extending along the lower leg with at the end a supporting element. This supporting element is supported by a correcting force at the lower leg. The correcting force results from an elastic tensioning of the lower leg orthosis.

### PRIOR ART

From AT 25 437A a lower leg orthosis is known, which is formed with a foot part and a coupling element. The foot part has a bearing element formed with a brace for a foot or shoe as well as a brace-like connecting element rigidly coupled to the bearing element, said connecting element extending upwardly from the bearing element. In the upper end region the connecting element is linked via a pivot joint to the coupling element. The pivot axis of the pivot joint is oriented approximately transversely to a walking direction or longitudinal axis of the foot and is positioned approximately at the height of the ankle joint. Owing to this orientation and arrangement of the pivot axis of the pivot joint a pivoting of the foot relative to the lower leg in the region of the ankle joint becomes possible when walking with the attached lower leg orthosis. In the end region lying opposite the pivot joint the coupling element is supported on the lower leg via a supporting element with a retaining device in the form of a band looped around the lower leg. In a front plane the coupling element and the connecting element form a vertical leg of an L, while the bearing element forms the horizontal leg of the L. Here the horizontal leg of the L and the vertical leg of the L do no form a right angle, but rather an acute or obtuse angle. The non-orthogonal arrangement of the legs of the L has the result that when the lower leg orthosis is subjected to a load, the weight of the person wearing the orthosis is transmitted in an end region of the horizontal leg of the L to the ground. On the other hand the weight of the person acts with a distance with a lever arm, for example centrally, on the horizontal leg of the L. This results in a correcting moment whose magnitude depends on the weight of the person and the lever arm. This correcting moment is counteracted by the support of the upper end region of the coupling element with supporting element and retaining device at the lower leg with the desired correcting force. In this connection the correcting force depends on the correcting moment and the length of the vertical leg of the L. For this configuration the correcting force is only produced when the lower leg orthosis is subjected to the weight of the person. The result is that the correcting force for the loading and load relief of the lower leg orthosis is subject to strong fluctuations when walking with the lower leg orthosis.

From DE 10 2010 019 355 A1 a lower leg orthosis is known, for which the correcting force should not be dependent on the loading, so that in the ideal case this force should be constant when walking. This should be achieved by a bending elasticity of the coupling element, which (in the case of a comparable construction to the embodiment according to AT 25437) should enable the orthosis to be applied under tension such that the supporting element, i.e. the horizontal leg of the L, is arranged parallel to the ground, whereas the coupling element is bent in such a way that it exerts the desired correcting force on the lower leg.

DE 103 38 128 B4 discloses a lower leg orthosis, in which the foot part is coupled rigidly and without a pivot joint to the coupling element. The coupling element forms in this case a semi-monocoque supporting element, which extends forwardly from the coupling element and with a circumferential angle of 180° around the lower leg. The supporting element can be adjusted in such a way before or when attaching the lower leg orthosis that the supporting element can be displaced forward or backward, so that in the end a matching of the angular position between the bearing element (i.e. the foot) and the supporting element (i.e. the lower leg) is thus possible. In addition to the semi-monocoque supporting element a retaining device in the form of a band is used, wherein the semi-monocoque supporting element and the band completely enclose the lower leg. Ends of the band are joined to one another via a hook-and-loop fastener. A pad or pelotte supported on the band or supporting element is used to cushion the contact surface between the lower leg orthosis and the lower leg.

With the known lower leg orthoses it is unavoidable that on prolonged wearing of the lower leg orthosis rubbing of a supporting element and/or a retaining device on the lower leg will occur, which adversely affects the wearing comfort of the lower leg orthosis.

DE 91 061 A relates to a non-generic orthosis, which supports an ankle joint in such a way that it cannot bend or twist sideways. The orthosis has a three-part splint or side bar, which is arranged on the rear side of the foot and lower leg. A first part and a second part of the splint are pivotally linked via a joint that is arranged above the ankle joint. The pivot axis of the joint has an orientation parallel to a pivot axis of the ankle joint, but is offset relative thereto by the arrangement of the splint on the rear side of the foot. The second part is connected to a third part of the splint, wherein the third part is displaceable relative to the second part in the longitudinal direction of the splint. A fixation belt is provided at the upper end of the splint, via which it is possible to fix the orthosis at the lower leg. Owing to the arrangement of the pivot joint and its orientation a pivoting of the foot relative to the lower leg when walking becomes possible. On the other hand due to the stiffness of the splint a lateral bending or twisting is to a limited extent prevented. Since the second part and the third part with the fixation belt arranged thereon are displaceable relative to one another in the longitudinal direction, a displacement of the position of the fixation belt on the lower leg during walking is prevented.

U.S. Pat. No. 6,792,700 B2 as well as U.S. Pat. No. 1,598,504 A also relate to non-generic orthoses for supporting an ankle joint so that the ankle joint cannot bend or twist sideways. With these orthoses the splints between the foot part and fixation belt are arranged behind the lower leg. These orthoses are in principle constructed similarly to the orthosis known from DE 91061 A. The orthoses also have a degree of freedom of movement and a degree of freedom of pivoting of parts of the splints relative to the foot parts, with which the same effect is achieved as in the case of the splint known from DE 91 061 A. There are only differences regarding the specific design of the foot parts and splints.

US 2007/0161934 A1 relates to a non-generic orthosis with a foot part and a splint extending along a shin-bone, i.e. in front of the lower leg. The foot part and the splint are connected to one another via connecting elements acting as a joint. At the end opposite the joint the splint is connected to a supporting element that comprises a fixation belt for fixing the orthosis at a lower leg. In order to be able to adapt the orthosis to different foot shapes and sizes, the connections between the foot part and the joint, the joint and the splint as well as the splint and the supporting element are formed as detachable connections with a degree of freedom of movement and pivoting. The joint has a pivot axis that is located in the region of a pivot axis of the ankle joint. In the joint damping elements are provided that counteract a pivoting about the pivot axis of the joint from a predetermined position. With a walking movement a pivoting of the foot relative to the lower leg about the pivot axis of the ankle joint back to the predetermined position should thereby be facilitated. The orthosis is in particular used to deal with so-called foot dorsiflexion weakness.

U.S. Pat. No. 3,827,430 A relates to a non-generic orthosis, which is also used to treat so-called foot dorsiflexion weakness. The orthosis has a foot part, which is pivotally connected to a splint, which extends along a calf, i.e. on a rear side of the lower leg. At its end facing away from the foot part the splint is connected to a supporting element, which comprises a fixation element in the form of a monocoque for fixing the orthosis to the lower leg. The splint and the supporting element are connected via a screw connection. For this purpose the splint has a longitudinal slit, in which a screw screwed to the supporting element is displaceably guided in the longitudinal direction of the splint. The foot part is elastically tensioned with respect to the splint in such a way that, when walking with the orthosis applied, a foot is always biased towards a predetermined position in which the foot is aligned roughly perpendicular to the lower leg in a plane containing the walking direction.

DE 601 16 607 T2 relates to a non-generic ankle-foot orthosis, which is produced from a plastic material. This orthosis has a foot part, a splint bearing against a lower leg and a fastening element for fastening the splint to the lower leg. The orthosis can be produced in several parts, which are connected to one another. In order to be able to adapt the orthosis to a lower leg before it is fixed at the lower leg, DE 601 16 607 T2 proposes making the connections detachable. After successfully adapting the orthosis the connections are fixed, whereby the orthosis can finally be used as intended.

### OBJECT OF THE INVENTION

The object of the present invention is to provide a lower leg orthosis that is improved as regards the wearing comfort, in particular as regards avoiding a rubbing during loading and load relief of the lower leg orthosis.

### Solution

The object of the invention is achieved according to the invention with the features of the independent claims. Further preferred embodiments according to the invention are the subject of the dependent claims.

### DESCRIPTION OF THE INVENTION

The present invention is based on a detailed investigation of the reasons that are responsible for rubbing of a supporting element on the lower leg when the lower leg orthosis is worn. These investigations have led to the result that, depending on the circumstances, with a correct attachment of the lower leg orthosis with a purely vertical loading and without pivoting movement of the foot part relative to the coupling element, no significant rubbing occurs. On the other hand in some cases there is increased rubbing if a pivoting of the foot part relative to the coupling element and thus a pivoting of the foot relative to the lower leg takes place, which for example is the case when walking. However, in other cases the same pivoting movement only leads to a rubbing with an at least significantly reduced extent. It was established by means of the investigations forming the basis of the invention that this apparently random different degree of rubbing can be due to the fact that the pivot axis of the pivot joint between the foot part and coupling element on the one hand, and the pivot axis of the ankle joint on the other hand, differ in different degrees from the ideal parallel and coaxial arrangement and orientation. Such a deviation, which is scarcely avoidable in the practical use of the lower leg orthosis, and whose extent may depend on the body geometry of the person wearing the lower leg orthosis, has the result that a contact surface of the lower leg moves on a (circular) path around the ankle joint, wherein this path deviates from the (circular) path of the counter-contact surface of the supporting element, which is predetermined by the pivot axis of the pivot joint. This deviation leads to a relative movement between the contact surface of the lower leg and the counter-contact surface of the supporting element, which is responsible for a compensatory movement, i.e. the rubbing. In addition it was established that the compensatory movement takes place in a direction that corresponds to the direction of the distance vector between the supporting element and foot part or the direction of the connecting straight line between the pivot joint and supporting element.

According to the invention these results are taken into account in that for the first time in a lower leg orthosis that is formed having a foot part, a coupling element pivotally connected to the foot part and a supporting element (which may also be formed integrally by the coupling element) supported on the coupling element, a degree of freedom of movement is provided. By means of this degree of freedom of movement, during use of the lower leg orthosis (i.e. not only when adapting the lower leg orthosis to different sizes or to facilitate an attachment of the lower leg orthosis) the distance between the foot part and the supporting element can be freely altered. In this way the different paths of the contact surface of the lower leg and the counter-contact surface of the supporting element can be compensated by the degree of freedom of movement, whereby a rubbing compensatory movement does not occur. In the context of the invention it is possible for the lower leg orthosis to provide exclusively the aforementioned degree of freedom of movement. However, it is also possible that at least one further degree of freedom of movement is present on the lower leg orthosis, for example in the walking direction and transverse to the first-mentioned degree of freedom of movement, and/or that further degrees of freedom of rotation are present.

The degree of freedom of movement for the distance between the foot part and the supporting element can be formed at an arbitrary point and between arbitrary structural elements of the lower leg orthosis. A freely alterable distance can be realised for example via the coupling element, in particular by a telescopic design of the coupling element. A further possibility is to provide the degree of freedom of movement between the coupling element and the foot part, in which this degree of freedom is integrated for example in a pivot joint between the coupling element and foot part. However, according to a preferred embodiment of the invention the degree of freedom of movement is formed between the supporting element and the coupling element. For some embodiments in this way the degree of freedom of movement can be realised with little effort. This type of implementation of the degree of freedom of movement also does not interfere with the design possibilities for further structural elements of the lower leg orthosis. For example, with this design there is no thickening or stiffening of a coupling element, as may be necessary if the coupling element is formed telescopically. A further possible advantage of the implementation of the degree of freedom of movement between the coupling element and the supporting element is that with a pivoting of the coupling element during use of the lower leg orthosis when walking, the direction of the degree of freedom of movement is also automatically changed, so that a compensatory movement enabled by the degree of freedom of movement is always oriented into the required direction.

Owing to the degree of freedom of movement created between the supporting element and the coupling element the supporting element and the coupling element are displaceable relative to one another. Here, a guide unit acting between the supporting element and the coupling element may be provided, which for example transfers forces acting transverse to the degree of freedom of movement between the coupling element and supporting element. The guide unit can be designed in any suitable way and can define arbitrary guide movements. For example, the guidance can take place along a curved path. However, preferably the guidance by the guide unit takes place along a linear guide path, which for example is advantageous if the required compensatory movement occurs substantially in a direction along the distance between the supporting element and foot part.

In principle it is possible that the degree of freedom of movement is not restricted. However, this might have the result that for example during storage, transportation or attachment of the lower leg orthosis, the supporting element might become detached from the coupling element. If this is undesirable, the degree of freedom of movement can be unilaterally restricted, preferably bilaterally limited. By limiting the degree of freedom of movement the lower leg orthosis is maintained as a unit when it is worn and also when it is attached or removed, whereby the use of the lower leg orthosis might be simplified.

In one embodiment according to the invention the degree of freedom of movement can be limited by a longitudinal slit in which a limiting element engages. The longitudinal slit can for example be formed in the coupling element, while the limiting element is provided on the supporting element. In a kinematic reversal the longitudinal slit may however also be provided in the supporting element, while the limiting element is then allocated to the coupling element.

The foot part of the lower leg orthosis can in principle be designed in any suitable way; see also the prior art discussed in the introduction. In a preferred design the foot part has a bearing element and a connecting element, which is arranged at an angle relative to the bearing element. The angle preferably approximately corresponds (in the tensioned state) to the angle between the foot surface and a side surface of the lower leg, which are accommodated by the lower leg orthosis.

In a preferred embodiment of the lower leg orthosis the bearing element and the connecting element of the foot part are formed as one part, in order to realise a simple and stable construction of the foot part. For example, the foot part can be produced from a blank, wherein the angled arrangement of the bearing element and connecting element can be achieved by bending a part of the blank designated for building the connecting element.

The foot part or the bearing element of the foot part can form a walking surface or shoe sole. In this way the foot part accommodates over a large area a foot surface of the foot accommodated by the foot part, whereby a good force transfer can be achieved, the wearing comfort can be improved, and the position and orientation of the lower leg orthosis can be predetermined.

In the lower leg orthosis according to the invention the foot part and the coupling element are connected to one another via a pivot joint approximately at the height of the angle joint. In this connection the pivot axis of the pivot joint is oriented approximately transverse to a walking direction or longitudinal axis of the foot. Owing to the pivot joint and its aforementioned orientation as well as arrangement, a pivoting of the foot relative to the lower leg in the region of the ankle joint should become possible when walking with the attached lower leg orthosis.

In order to improve the retention of the supporting element of the lower leg orthosis independently of the varying load on the lower leg carrying the lower leg orthosis, in a further embodiment the invention proposes that the support surface is connected to a retaining device, which at least partly surrounds the lower leg. In order simply to mention a non-limiting example; the retaining device can be formed with a monocoque. It is also possible to build the retaining device with a band that at least partly loops around the lower leg and that for example can be adjusted as regards its length via a hook-and-loop fastener.

Preferably the lower leg orthosis can be elastically tensioned. Owing to the elastic tensioning of the lower leg orthosis a lateral correcting force is exerted on the lower leg via the supporting element. The tensioning can in this connection lead for example to a correcting force that is permanently exerted by the worn lower leg orthosis (see in particular DE 10 2010 019 355 A1). It is however also possible that the tensioning (and thus the correcting force) depends on a loading of the worn or attached lower leg orthosis (see in particular AT 25 437).

In a preferred embodiment of the lower leg orthosis according to the invention the correcting force of the attached lower leg orthosis exerted by the tensioning depends on the loading of the foot part: without support of the supporting element at the lower leg the loading of the foot part resting on the ground would then lead to a lateral misalignment in a frontal plane. If however the supporting element when loaded is supported at the lower leg, this aforementioned misalignment is eliminated. In this case the elimination of the misalignment that would occur without elastic tensioning of the lower leg orthosis and if the supporting element were not supported, results in a correcting force exerted on the lower leg.

The lower leg orthosis according to the invention can be designed for a left lower leg as well as for a right lower leg. Preferably the supporting elements and the coupling elements for a left and a right lower leg are identical. According to an embodiment of the invention blanks for the foot parts can be designed identically. In this way the proportion of identical parts for the production of different lower leg orthoses for a left and a right lower leg can be increased. The finished shaped foot parts then differ only by the direction into which the blank for a foot part the connecting element is deformed relative to the bearing element. In the case of a multi-part foot part that is produced with separate connecting and bearing elements, the direction into which the connecting element is attached to the bearing element can be reversed for a lower leg orthosis for the right and left lower leg.

Advantageous modifications of the invention are disclosed in the claims, description and drawings. The advantages of features and of combinations of several features mentioned in the introduction to the description are given simply by way of example and can be used alternatively of cumulatively, without the advantages necessarily having to be achieved by embodiments according to the invention. Without thereby altering the subject matter of the accompanying claims, the following applies as regards the disclosure of the original application documents and patent: further features can be derived from the drawings—in particular from the illustrated geometries and the relative dimensions of several structural parts with respect to one another as well as their relative arrangement and working connection. The combination of features of different embodiments of the invention or of features of different claims is also possible by way of variation from the selected mutual dependencies of the claims and is hereby promoted. This also relates to those features that are illustrated in separate drawings or that have been mentioned in their description. These features may also be combined with the features of different claims. Similarly, features specified in the claims can be omitted in further embodiments of the invention.

The features mentioned in the claims and the description should be understood in terms of their number in such a way that exactly this number or a larger number than the specified number may be present, without the need for an explicit use of the adverb “at least”. If therefore for example one connecting element is discussed, then this should be understood to mean that just one connecting element, two connecting elements or more connecting elements may be present.

### BRIEF DESCRIPTION OF THE DRAWINGS

The invention is discussed and described in more detail hereinafter with the aid of preferred embodiment illustrated in the drawings.

FIG. 1 shows a lower leg orthosis in a three-dimensional view.

FIG. 2 shows the lower leg orthosis according to FIG. 1 in a side view.

FIG. 3 shows the lower leg orthosis according to FIGS. 1 and 2 in a view from behind.

FIG. 4 shows the lower leg orthosis according to FIGS. 1 to 3 in a plan view.

FIG. 5 shows the lower leg orthosis according to FIGS. 1 to 4 in the attached or worn state in a side view.

FIG. 6 shows the lower leg orthosis according to FIGS. 1 to 5 in the attached or worn state in a view from behind.

FIG. 7 shows the kinematics of a lower leg orthosis without the degree of freedom of movement according to the invention.

FIG. 8 shows in a three-dimensional exploded view a supporting element as well as a coupling element of a lower leg orthosis according to the invention.

FIG. 9 shows in a three-dimensional view the coupling element and supporting element according to FIG. 8 in the assembled state, here in a first limited end position of the degree of freedom of movement.

FIG. 10 shows in a three-dimensional view the coupling element and supporting element according to FIG. 8 in the assembled state, here in a first limited end position of the degree of freedom of movement.

FIG. 11 shows a section XI-XI through the coupling element and the supporting element according to FIG. 10.

### DESCRIPTION OF THE FIGURES

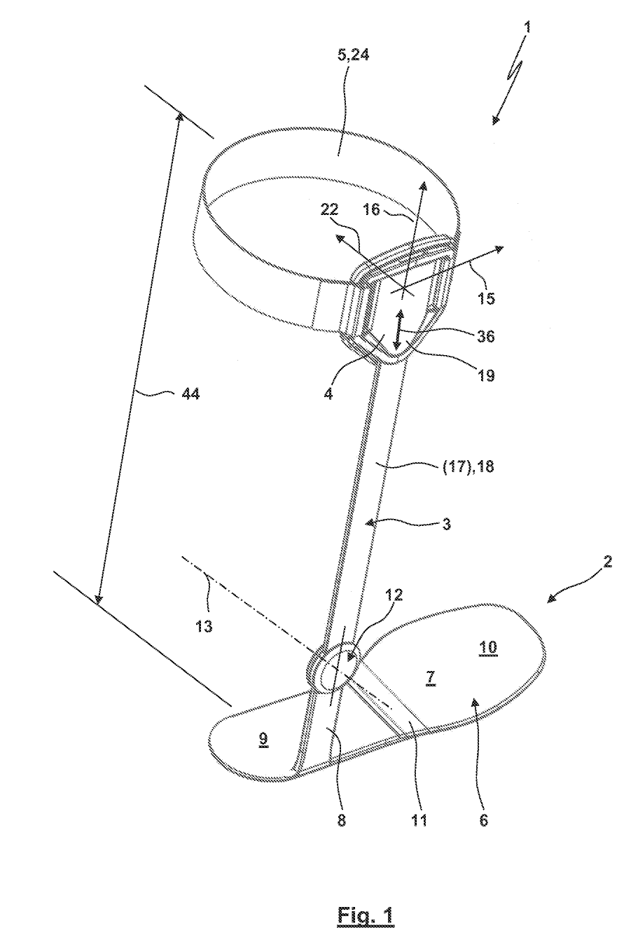

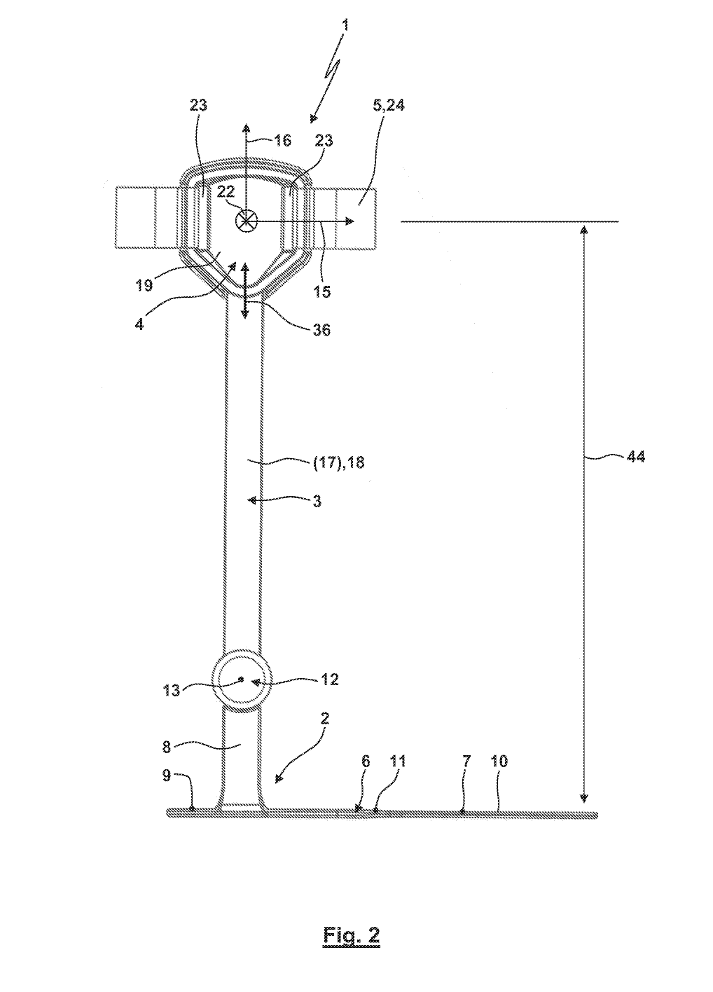

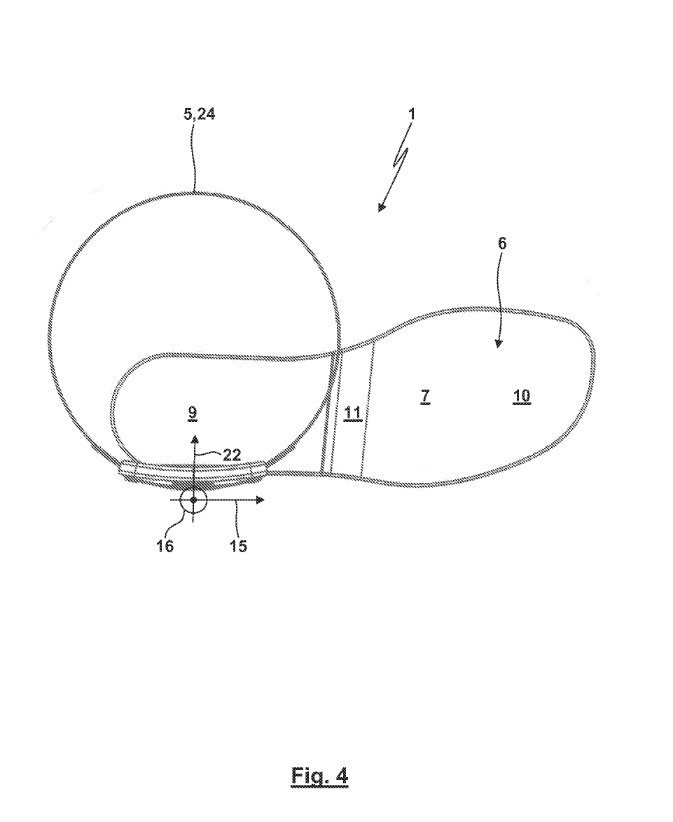

FIGS. 1 to 4 show different views of a lower leg orthosis 1 according to the invention. The lower leg orthosis 1 has a foot part 2, a coupling element 3, a supporting element 4 and a retaining device 5.

The foot part 2 is formed with a bearing element 6, on which the foot rests or is supported when the lower leg orthosis 1 is worn. The bearing element 6 can in this connection be designed in the form of a brace, exactly in accordance with the prior art AT 25437. For the embodiment illustrated here the bearing surface 6 is however formed over a large area with a walking surface 7, which corresponds in the manner of a shoe sole largely to the shape of the underneath of the foot, wherein the walking surface 7 can also be correspondingly contoured. In addition the foot part 2 has a connecting element 8. The connecting element 8 extends approximately vertically to the walking surface 7, so that this is oriented approximately parallel to the lower leg of the person wearing the lower leg orthosis 1. In this connection “approximately vertically” is also understood to include a deviation from an angle of 90°, for example by +/−10° or +/−20°, in an arbitrary spatial direction, this deviation resulting from the fact that the connecting element 8 (and the coupling element 3) have to go over the outside of the lower leg, for example also over a projecting ankle joint. The connecting element 8 is in this connection formed as a brace. The connecting element 8 and the bearing element 6 are rigidly connected to one another, so that the connecting element 8 projects from the bearing element 6. For the illustrated embodiment the bearing element 6 and the connecting element 8 are formed in one piece. Preferably the foot part 2 is formed as a composite body in the required partial regions in order to stiffen it. For example, a steel insert extends in a heel region 9 as well as in the region of the connecting element 8 in the foot part 2. The insert maintains the angle between the connecting element 8 and bearing element 6, provides a rigid bearing surface for the heel of the foot, and should also avoid a bending of the heel region 9 about a longitudinal axis, which could lead to a tilting of the foot. On the other hand a front foot region 10 is formed without a steel insert, so that this is in particular elastic with respect to a bending about a transverse axis, in order to allow a rolling movement when walking. The foot part 2 is produced for example by an injection moulding process, in which the steel insert is moulded in with a suitable plastic material. It is of course also possible that in order to form the foot part 2, a separately produced sole is bonded onto a steel support. It is furthermore possible that an insert of the composite body is formed with carbon mats, whereby a foot part of reduced weight can be formed. In FIG. 1 it can be seen that as regards the steel inlay extending only in the heel region 9 of the bearing element 6, the greater thickness around the heel region 9 over a transition region 11 transforms into the front foot region 10, which has a smaller thickness.

Without this necessarily being the case, a production of the foot part 2 for a foot part 2 for a right foot as well as for a foot part 2 for a left foot can be simplified and standardised by first of all identically producing a blank for the bearing element 6 and the connecting element 8 both for a right foot part 2 and also for a left foot part 2, with a flat alignment of the connecting element 8 with the bearing element 6. Depending on whether such a blank is then to be used for a right or left foot part 2, the connecting element 8 is then offset at an angle upwards or downwards by cold or hot deformation, wherein then for the right and left foot part 2 different sides of the bearing element 6 can be used as the surface contacting the foot.

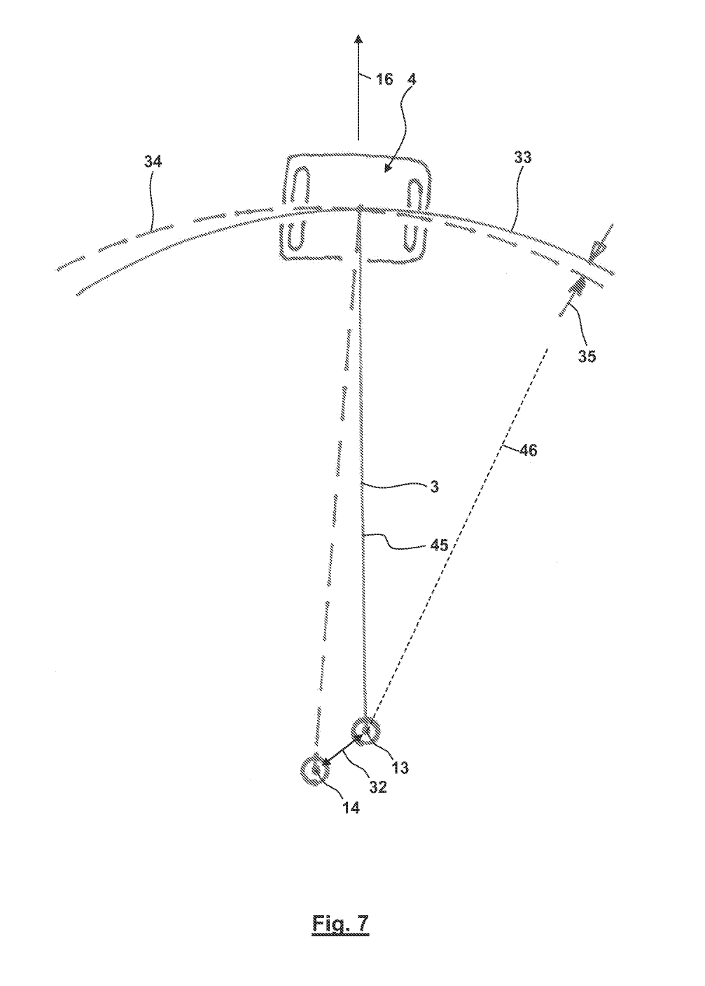

A connection between the connecting element 8 and thus the foot part 2 and the coupling element 3 takes place via a pivot joint 12, which defines a pivot axis 13. The pivot joint 12 is configured in a manner known per se, this preferably being designed structurally in such a way that it avoids damaging the skin when it pivots about the pivot axis 13 and is formed grease-free or is encapsulated to prevent leakage of grease. The arrangement and orientation of the pivot joint 12 takes place in particular by choosing and orienting the connecting element 8 so that, in the ideal case, the pivot axis 13 is oriented coaxially to a pivot axis 14 of the ankle joint. The pivot axis 13 is preferably arranged approximately in the horizontal plane and extends transverse to a walking direction 15. In the pivot position illustrated in FIG. 1 or neutral position of the pivot joint 12 the coupling element 3 forms a continuation of the connecting element 8 in the direction of an upper axis 16. An upper axis 16 denotes hereinafter an axis that is oriented approximately vertically to the bearing element 6 and whose alignment is independent of the pivoting of the coupling element 3 relative to the foot part 2. On the other hand a coupling axis 46 denotes an axis that passes through the longitudinal axis of the coupling element 3 and is oriented approximately parallel to the lower leg of the person wearing the lower leg orthosis 1, so that the orientation of this coupling axis 46 changes with the pivoting of the coupling element 3 (see also FIG. 7). The connecting element 3 connects the pivot joint 12 to the supporting element 4. The coupling element 3 is formed to be flexurally elastic about a bending axis that is oriented parallel to the walking direction 15. Here, the coupling element 3 is in the illustrated embodiment formed in the manner of a brace. Different embodiments are also possible in this case, see for example DE 10 2010 019 355 A1, where the coupling element is formed with flexurally elastic tubes 8. For example, the coupling element 3 is formed with a stainless steel brace 17 of constant rectangular cross-section over the longitudinal axis from a band-shaped raw material, which is provided with a tubular or sprayed-on sheathing 18 of an elastic material that does not irritate the skin. In end region facing the pivot joint 12 the supporting element 4 is held at the coupling element 3 in a manner specified in more detail hereinafter.

The supporting element 4 is to a broad approximation formed in the shape of a plate and is produced for example from plastic material, this preferably having a certain curvature and/or elasticity in order to allow an adaptation to the lower leg of the person wearing the lower leg orthosis 1. It is furthermore possible that the supporting element 4 is formed with a base body 19 on which a cushion element 20 is held, which allows for an elastic and gentle transfer of a correcting force 21 in a correcting force direction 22 onto the lower leg. The correcting force direction 22 is oriented transverse to the walking direction and approximately parallel to the pivot axis 13 as well as transverse to the bending axis of the coupling element 3. The supporting element 4 has in the region of the base body 19 recesses 23 or slits arranged with respected to the circumferential direction on the edge around the lower leg, through which a band 24 forming the retaining device 5 can be looped, which can be fastened for example by a hook-and-loop fastener or other type of closure or a clamping means tightly around the lower leg of the person wearing the lower leg orthosis 1.

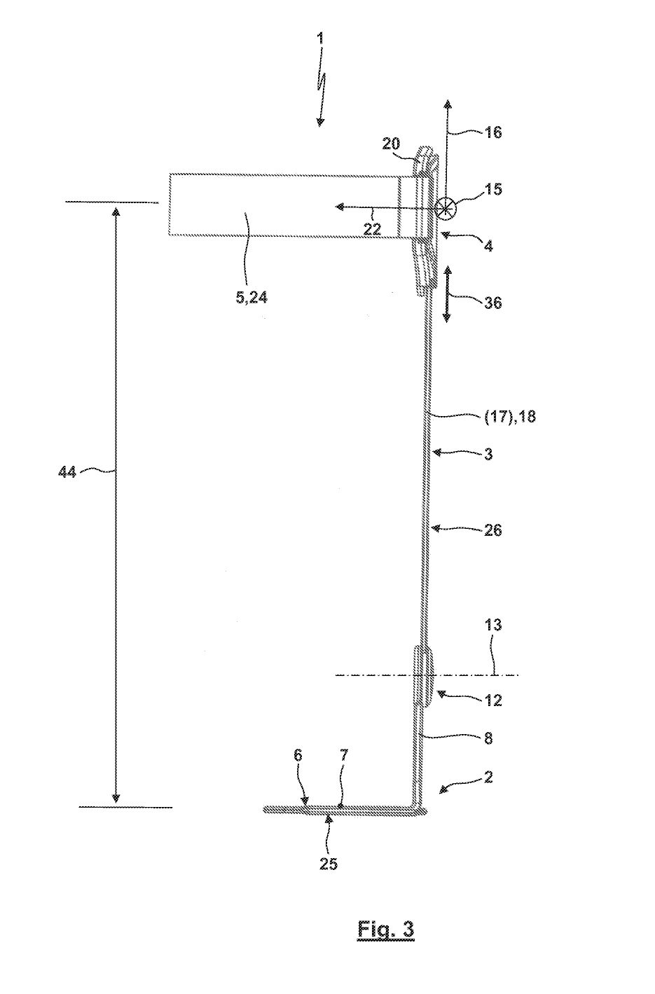

In FIG. 3 it can be seen that the foot part 2 and the coupling element 3 are formed L-shaped in a front view (or here in a rear view), wherein a horizontal leg 25 of the L is formed by the bearing element 6, while the vertical leg 26 is formed with the connecting element 8, the pivot joint 12 and the coupling element 3. Depending on the circumstances, the vertical leg is after each pivoting of the pivot joint 12 vertically offset relative to the plane of the drawing according to FIG. 3.

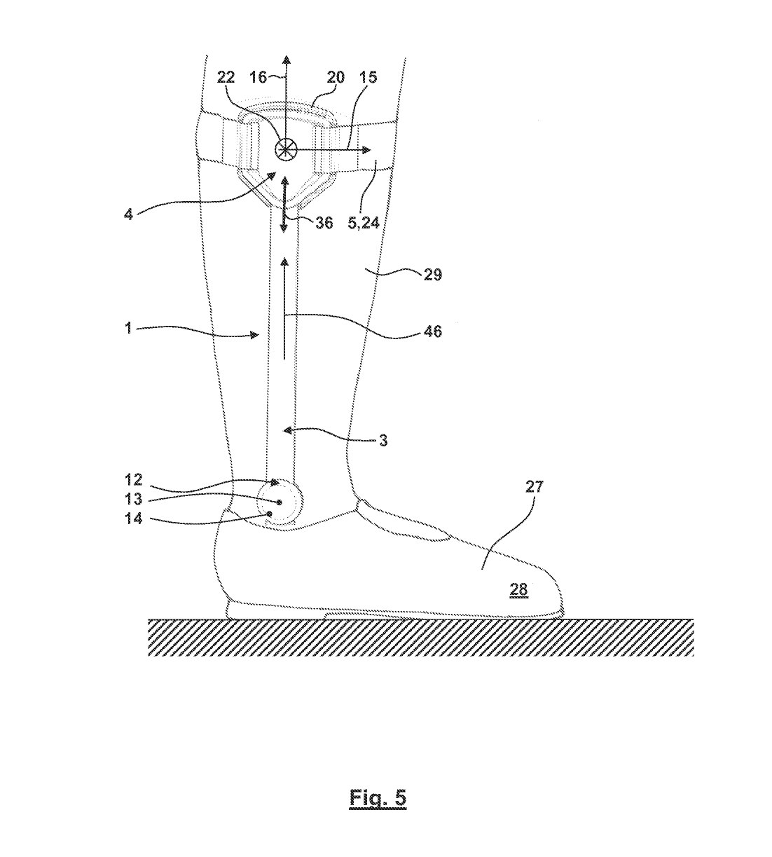

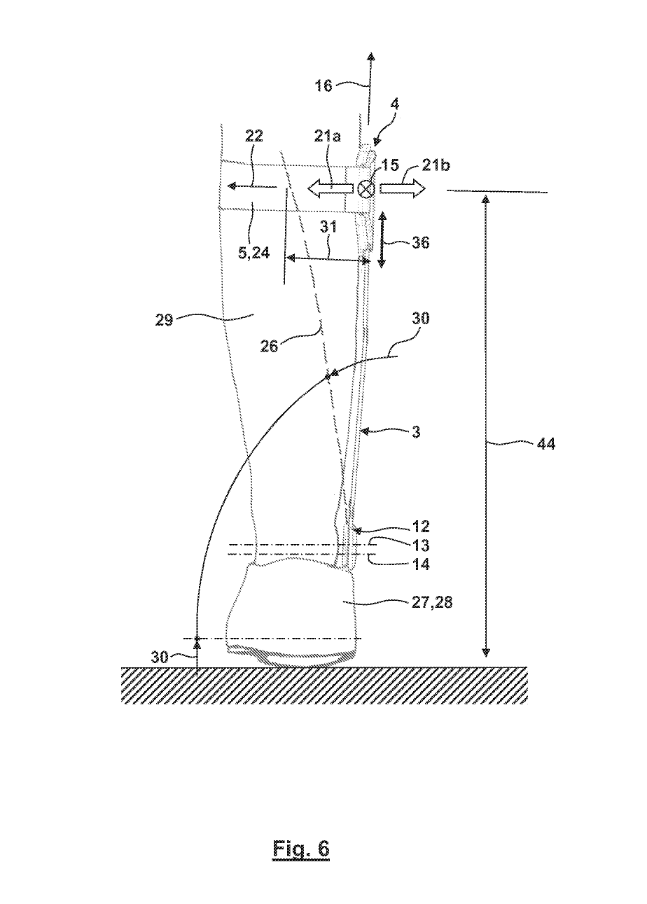

FIGS. 5 and 6 show the attached lower leg orthosis 1, for which a foot 27 is supported or rests on the bearing element 6. The foot part 2 is largely arranged in a shoe 28, wherein in the illustrated embodiment the upper end region of the connecting element 8 with the pivot joint 12 projects from the shoe 28 (although this is not necessarily the case). The coupling element 3 extends laterally along a lower leg 29. The retaining device 25 formed with the band 24 is looped tightly around the lower leg 29, so that a connection that is as slip-resistant as possible is formed between the supporting element 4, base body 19, cushion element 20 and retaining device 5 as well as the band 24.

In FIGS. 1 to 4, in order to simplify the illustration and as a consequence of the limited drawing capacities, the angle of the longitudinal axis of the connecting element 8 and of the coupling element 3 (i.e. of the leg 26) relative to the bearing element 6 (i.e. to the leg 26) in projection onto the plane that is bounded by the upper axis 16 and the correcting force direction 22, is shown as a right angle. In fact, in the unloaded state of the lower leg orthosis 1 an acute or obtuse angle 30 is formed, as was discussed for the prior art mentioned in the introduction. In FIG. 6 the undeformed, force-free state of the leg 26 is shown diagrammatically by the dashed line. Of course it is also possible that the leg 26 in the plane in FIG. 6 or in a projection into this plane is curved. In any case an offset 31 exists for the leg 26 in the region of the supporting element 4, which for the embodiment illustrated in FIG. 6 is oriented inwardly in the direction of the lower leg 29. In order to allow a worn or attached state of the lower leg orthosis 1 according to FIG. 6, the leg 26 must be deformed outwardly by an elastical bending by a correcting force 21b, i.e. the offset must be reduced to zero. This correcting force 21b acting on the leg 26 is caused by the lower leg 29, so that a correcting force 21 acts on the lower leg 29. According to the prior art discussed in the introduction, different basic principles are possible for producing the correcting force 21a, wherein these can for example act permanently on attachment of the lower leg orthosis 1, or can arise only when the lower leg orthosis 1 is loaded by the weight of the person wearing the lower leg orthosis 1.

As previously discussed, in the ideal case the pivot axis 13 of the pivot joint 12 and also the pivot axis 14 of the ankle joint are arranged coaxially to one another. This is however in practice not always the case. The reasons for this are for example the following:
different body sizes of the persons wearing the lower leg orthosis 1,
an incorrect attachment of the lower leg orthosis 1, and/or
changes of the orientation and position of the pivot axis 14 of the ankle joint, for example during a walking movement with a pivoting of the ankle joint or depending on the load.

A temporary or permanent distance 32 of the pivot axes 13, 14 is illustrated by way of example in FIG. 7. If the supporting element 4 is fixed in the direction of the coupling axis 46 to the coupling element 3 (so differing from the invention), then the supporting element 4 with the counter-contact surface formed with it for the lower leg moves about the pivot joint 12 and the pivot axis 13 moves on a circular path 33, which is illustrated by a continuous line in FIG. 7. On the other hand the associated contact surface of the lower leg 29 moves on a circular path 34 about the ankle joint and about the pivot axis 14, the circular path 34 being shown by the dashed line in FIG. 7. The different circular paths 33, 34 consequently mean that, depending on the pivot angle between the contact surface of the lower leg and the counter-contact surface formed by the supporting element 4 or cushion element 20, a displacement 35 takes place in the direction of the coupling axis 46, which according to the prior art can be compensated only by a slipping of the supporting element 4 or cushion element 20 relative to the skin of the lower leg 29. This slipping compensatory movement is according to the invention at least to some extent avoidable due to the fact that the lower leg orthosis 1 has a degree of freedom of movement 36. With the pivot angles of the pivot joint 12 that normally occur in use, it is in some cases sufficient if the degree of freedom of movement 36 is oriented in the direction of the upper axis 16, i.e. the degree of freedom does not move in conjunction with the pivoting of the coupling element 3. Thus in principle it is also possible that the degree of freedom of movement is integrated into the coupling region between the coupling element 3 and connecting element 8 and into the pivot joint 12. According to the invention it is however preferably proposed that the degree of freedom of movement 36 is provided between the coupling element and the supporting element 4, so that the degree of freedom of movement 36 changes its direction with the pivoting of the coupling element 3 and a complete compensation of the displacement 35 (and not simply the compensation of the significant component) can take place. The degree of freedom of movement 36 is symbolised by an arrow in FIGS. 1 to 6 and is oriented parallel to the coupling axis 46.

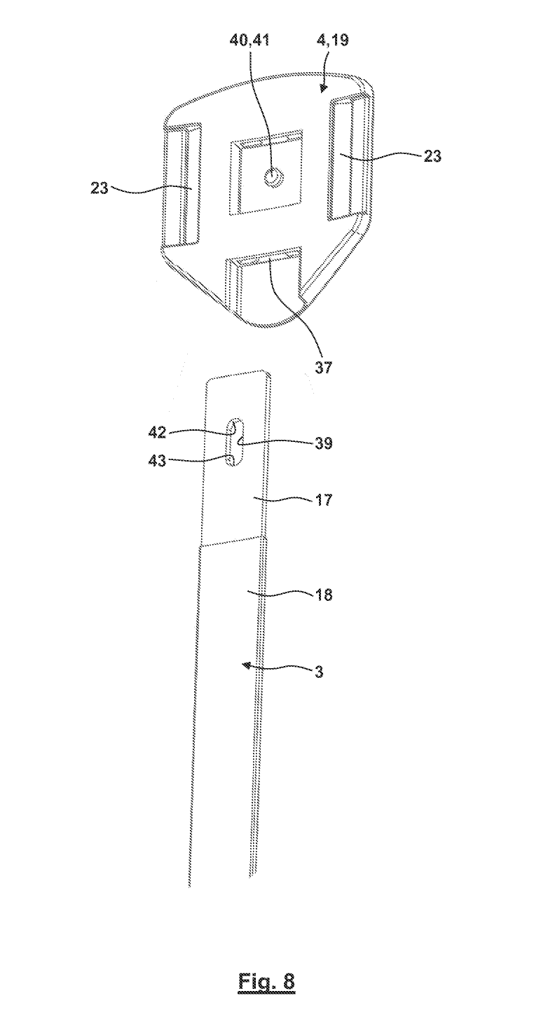

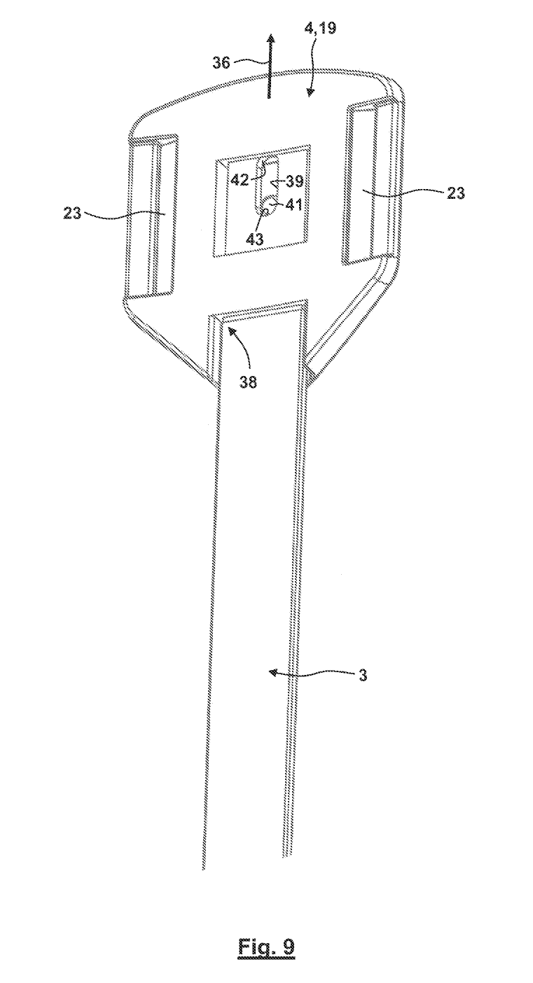

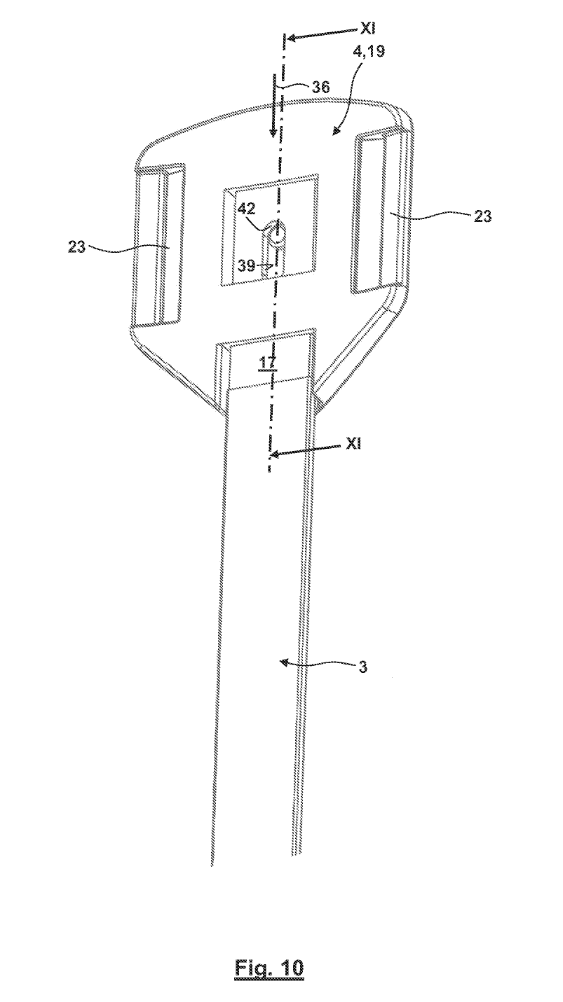

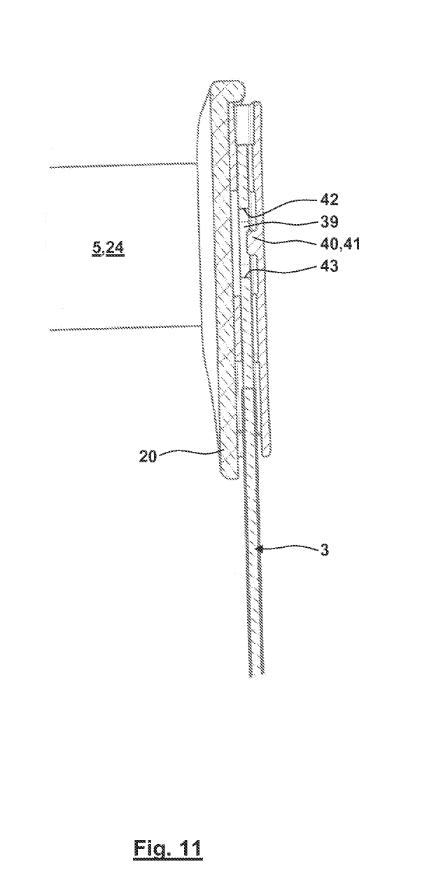

FIGS. 8 to 11 show an exemplary structural embodiment for allowing such a degree of freedom of movement 36 between the coupling element 3 and the supporting element 4, without the invention being restricted to this specific structural configuration. The supporting element 4 has a recess 37, which at least in a longitudinal section is formed with a closed edge with a cross-sectional surface that forms a transition fit or clearance fit with the cross-section of the coupling element 3, here the stainless steel brace 17. Accordingly an end region of the coupling element 3, here the stainless steel brace 17 (which in this region is not provided with a sheathing 18) can be introduced into the recess 37. The closed edge recess 37 and the cross-section of the coupling element 3, here the stainless steel brace 17, form a guide unit 38, which allows a relative sliding movement between the supporting element 4 and coupling element 3 in the direction of the longitudinal axis of the coupling element 3, while the guide unit 38 prevents relative movements transverse to this guide direction and transmits forces transverse to this guide direction, in particular the correcting force 21. In the end region which in the installed state extends into the recess 37 (in this case the stainless steel brace 17 without sheathing 18) the coupling element 3 has a longitudinal slit 39 in which a limiting element 40 (in this case a pin 41) engages. The longitudinal extension of the longitudinal slit 39 limits the maximum possible displacement path of the degree of freedom of movement 36. Limit stops 42, 43, which are formed with the end regions of the longitudinal slit 39, abut in the end region of the degree of freedom of movement 36 against the limiting element 40. By means of the degree of freedom of movement 36 the radius 45, on which the supporting element 4 moves along the circular path 33, can change automatically during the pivoting about the pivot joint 12. Via the degree of freedom of movement 36 a distance 44 between the bearing element 6 and the supporting element 4 can also change during use of the lower leg orthosis 1, i.e. when walking with attached lower leg orthosis 1.

An assembly of the coupling element 3 in the supporting element 4 can, in the case of a one-piece formation of the supporting element 4, be made possible by an elasticity of the latter, by means of which the limiting element 40 can be forced outwardly so that the coupling element 3 can be inserted into the recess 37, until the limiting element 40 “snaps” into the longitudinal slit 39. It is also possible that, in order to allow the installation the supporting element 4 is formed at least in two parts or the limiting supporting element 40 is an additional bolt to be passed through the supporting element 4, which is then subsequently inserted through the supporting element 4 and the longitudinal slit 39 and secured in place. For example, as a result of the limit stops 42, 43 the length of the degree of freedom of movement 36 is 1 cm or 2 cm, though deviations of +/−50% or 20% of these lengths are also possible. Preferably the lower leg orthosis 1 is designed so that with a vertical alignment of the coupling element 3 the limiting element 40 is located in the middle of the longitudinal slit 39. It is however also possible that (with a single “rubbing” movement) the supporting element 4 moves to the correct position during take-up of the use of the lower leg orthosis 1. It is understood that, as a variation of the illustrated embodiment, a limiting of the degree of freedom of movement 36 can simply take place unilaterally or even cannot take place at all. It is possible that, in addition to the degree of freedom of movement 36, further degrees of freedom in the lower leg orthosis 1 and in particular between the coupling element 3 and supporting element 4 are present, which for example can be an additional degree of freedom of movement in the walking direction or a degree of freedom of rotation about the correcting force direction 22. In certain cases the lower leg orthosis 1 is also provided with a certain degree of flexibility, for example in the region of the band 24, the cushion element 20 and/or the supporting element 4. In addition to the illustrated and described connection between the band 5 and supporting element 4, any other suitable retaining devices and connection possibilities can be employed, for example semi-monocoque retaining devices, connections of a band by means of a clip, etc.

The component parts of the lower leg orthosis 1 can be produced for example at least partly in an injection moulding process, in which also if necessary for reinforcement purposes inserts, for example steel sheets, carbon mats, etc., can be moulded in. Any suitable plastic material can be used in the injection moulding process, wherein this material may also be porous and expanded by using a foaming agent. Possible useable plastics are for example PU, TPE, TPU. It is possible that, by way of variation from the illustrated embodiments, the longitudinal slit 39 is not formed rectilinearly but is curved, whereby a further compensatory movement in the walking direction 15 could take place with automatic coupling of the compensatory movements via the degree of freedom of movement 36 and the additional compensatory movement in the walking direction 15. In addition to the use of an injection moulding manufacturing process also a stamping production method can be employed. Preferably the production of the illustrated supporting element 4 with the recess 37 is carried out by using a core, which serves to shape the recess 37. For this purpose a core slide bar can also be used, which can also be sub-divided in the mid-plane of the limiting element 40.

In order to allow a good sliding movement between the supporting element 4 on the one hand and the coupling element 3 on the other hand, a plastic material with a filler that promotes a sliding movement is preferably used for the supporting element 4. For example, PA materials can be used for this purpose with a filler in the form of Teflon, graphite, etc. The choice of the fillers is made depending on the desired mechanical properties and strength requirements. It is also possible to use a plastic POM or PP. Furthermore for the cushion materials it is possible to use a PE foam. Preferably contact regions of the lower leg orthosis 1, i.e. in particular the supporting element 4 and/or the coupling element 3, are covered with velour, in order to improve the compatibility with the skin. It is possible to effect the connection between the base body 19 and the cushion element so that the base body 19 is covered with velour on the side facing the lower leg 29, while the cushion element 20 is provided on its rear side with hook-and-loop fasteners, so that the connection between the base body 19 and cushion element 20 can take place via a hook-and-loop connection with hooks in the velour of the base body 19.

Preferably the lower leg orthosis 1 is produced in different sizes that take account of different body sizes of persons wearing the lower leg orthosis 1. In this connection the lower leg orthosis 1 can for example be produced with lengths of the connecting element 8 of 7 cm, 8 cm, 9 cm and 10 cm (preferably +/−20%).

The length of the coupling element preferably varies between 270 mm and 375 mm (deviations of +/−20% being possible) for the different body sizes.

The degree of freedom of movement 36 can alternatively or in addition to the compensation of the displacement 35 according to FIG. 7 be used to compensate a movement of the lower leg orthosis 1 in the shoe, which occurs when wearing and during the loading and load relief of the lower leg orthosis 1.

The person skilled in the art in orthopaedics will recognise that the angular relationships chosen in the present description, such as perpendicular, transverse, coaxial, parallel, simply denote the approximate alignments, and can vary depending on
the body geometry of the wearer of the lower leg orthosis 1,
the exact position and dimensions of the foot and lower leg, and also
bulges for example of the ankle joint
   and also on the movement.

It is also possible that structural elements described as being flat or rectilinear can in fact also be formed bent or curved.

I claim:

1. Lower leg orthosis with
a) a foot part,
b) a coupling element pivotably connected to the foot part and
c) a supporting element supported at the coupling element
d) wherein the coupling element extends laterally along a lower leg carrying the lower leg orthosis
e) wherein the foot part is connected via a pivot joint to the coupling element, wherein a pivot axis of the pivot joint is arranged coaxially to a pivot axis of an ankle joint or a deviation from the coaxial arrangement and orientation depends on the body geometry of the person wearing the lower leg orthosis,
f) wherein the lower leg orthosis can be elastically tensioned, whereby a lateral correcting force is exerted on the lower leg, wherein the elastical tension of the lower leg orthosis and the lateral correcting force depends on a load of the lower leg orthosis, and
g) wherein a degree of freedom of movement is provided, via which during use of the lower leg orthosis a distance between the foot part or a pivot joint connecting the foot part to the coupling element and to the supporting element is freely alterable, wherein the degree of freedom of movement is used for a compensation of a displacement resulting from the load of the lower leg orthosis.

2. Lower leg orthosis according to claim 1, characterised in that the degree of freedom of movement is formed between the supporting elements and the coupling element.

3. Lower leg orthosis according to claim 2, characterised in that a guide unit acts between the supporting element and coupling element.

4. Lower leg orthosis according to claim 1, characterised in that the degree of freedom of movement is limited at least unilaterally.

5. Lower leg orthosis according to claim 4, characterised in that a longitudinal slit is provided to limit the degree of freedom of movement.

6. Lower leg orthosis according to claim 1, characterised in that the foot part is formed with a bearing element and with a connecting element extending at an angle relative to the bearing element.

7. Lower leg orthosis according to claim 6, characterised in that the bearing element (6) and the connecting element (8) are formed in one piece.

8. Lower leg orthosis according to claim 1, characterised in that the foot part or the bearing element forms a walking surface or shoe sole.

9. Lower leg orthosis according to claim 1, characterised in that the supporting element is connected to a retaining device, which at least partly surrounds the lower leg carrying the lower leg orthosis.

10. Lower leg orthosis according to claim 1, characterised in that with the foot part resting on the ground and the foot resting on the foot part
a) without support of the supporting element at the lower leg the supporting element is arranged in a frontal plane laterally with an offset relative to the lower leg, and
b) with support of the supporting element on the lower leg the correcting force acting on the supporting element results from the elimination of the offset with an elastic tensioning of the lower leg orthosis.

11. Lower leg orthoses according to claim 1, with
a) a first group of lower leg orthoses for a right lower leg and
b) a second group of lower leg orthoses for a left lower leg, wherein
c) the lower leg orthoses of the first and second groups are produced with the same blanks for the foot parts, and
d) the foot parts of the lower leg orthoses of the first and second groups differ by the direction into which the connecting element of the blank for a foot part is deformed relative to the bearing element.
